(19) United States
(12) Patent Application Publication
(10) Pub. No.: US 2010/0176214 A1
(43) Pub. Date: Jul. 15, 2010

(54) GREETING CARD FRAGRANCE DELIVERY SYSTEM

(76) Inventors: Ashok V. Joshi, Salk Lakt City, UT (US); Truman Wold, Salk Lake City, UT (US)

Correspondence Address: CERAMATEC, INC., 2425 SOUTH 900 WEST, SALT LAKE CITY, UT, 84119, US

(21) Appl. No.: 12/686,293

(22) Filed: Jan. 12, 2010

Related U.S. Application Data
(60) Provisional application No. 61/144,356, filed on Jan. 13, 2009.

(51) Int. Cl. A61L 9/04

(52) U.S. Cl. 239/57

(57) ABSTRACT

A fragrance delivery system for delivering a fragrance from a card is disclosed. In the system, a canister attached to a first surface, which may be the face of a card. An emanator is disposed within the canister, including a fragrance-emitting material. A removable cover is attached to a second surface, which may be an opposing face of the card, such that the when the first and second surfaces are brought together, the cover inhibits or prevents fragrance from escaping the canister. Upon opening the card, the cover is removed and fragrance escapes from the canister.

### RELATED APPLICATIONS

This application claims priority to U.S. Provisional Patent No. 61/144,356 filed on Jan. 13, 2009 and entitled Greeting Card Fragrance Delivery System.

### FIELD OF THE INVENTION

The present invention relates in general to methods and systems for delivering fragrance from a greeting card or other paper or substantially thin housing over an extended period of time or use.

### BACKGROUND

The greeting card business is a multi-billion dollar business, based on total unit sales of 6 billion greeting cards for the year. It is a highly competitive market with multiple corporate and home based businesses. Most cards contain a message coupled with a graphic design or picture. However this is not always enough to distinguish one card from another in the mind of consumer. Recently, card manufacturers have added an audible aspect to greeting cards and other stationary-type products. However, there is a need for makers of greeting cards and stationery products to distinguish their products further in a unique way and maintain that uniqueness over the shelf life of the product.

### SUMMARY OF THE INVENTION

In response to needs in the industry, a fragrance delivery system is disclosed herein to provide an olfactory element to a card or paper product. The fragrance delivery system comprises a canister attached to a first surface. The canister has an orifice disposed threrein. An emanator may be disposed within the canister. The emanator contains a fragrance-emitting material. A removable cover disposed with an second surface such that the when the first and second surfaces are brought together, the cover covers one or more of the orifices disposed within the canister, thereby inhibiting or substantially preventing a fragrance in the fragrance-emitting material from escaping the canister. The cover and canister may be substantially flat and each may be positioned in opposing sides of a card or paper product. The term “card” or “greeting card” as used herein shall include paper products, stationery items and other products. The cover may be configured to cover one or more orifices of the canister to prevent or partially inhibit fragrance from emanating from the canister. In one embodiment, the cover is magnetized such that it covers the canister until the force of opening the card removes the cover.

In one embodiment, the fragrance-emitting material is a liquid. The emanator may be an absorbent material such as paper, gelatinous substances, plastic, cellulous material and the like. For example, the emanator and fragrance-emitting material may be paper soaked in a fragrant liquid. In one embodiment, the emanator is a porous material configured such that the surface tension and/or wicking action within the emanator 24 is equal to or greater than the force of gravity, which allows the emanator 24 to keep the fluid from dripping or seeping out.

### BRIEF DESCRIPTION OF THE SEVERAL VIEWS OF THE DRAWINGS

In order that the manner in which the features and advantages of the invention are obtained will be readily understood, a more particular description of the invention will be rendered by reference to specific embodiments thereof that are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a perspective view of a single fold card that could embody a fragrance delivery system according to the present invention;

FIG. 2 is a perspective view of the card of FIG. 1, partially opened to reveal one embodiment of the fragrance delivery system of the present invention;

FIG. 3 is a perspective view of the card of FIG. 2;

FIG. 4 is a perspective cutaway view of the card of FIG. 2;

FIG. 5 is a perspective view of the card of FIG. 1, partially opened to reveal another embodiment of the fragrance delivery system of the present invention;

FIG. 6 is a perspective cutaway view of the card of FIG. 5; and

FIG. 7 is a perspective cutaway view of another embodiment of the fragrance delivery system incorporated into card.

### DETAILED DESCRIPTION OF THE INVENTION

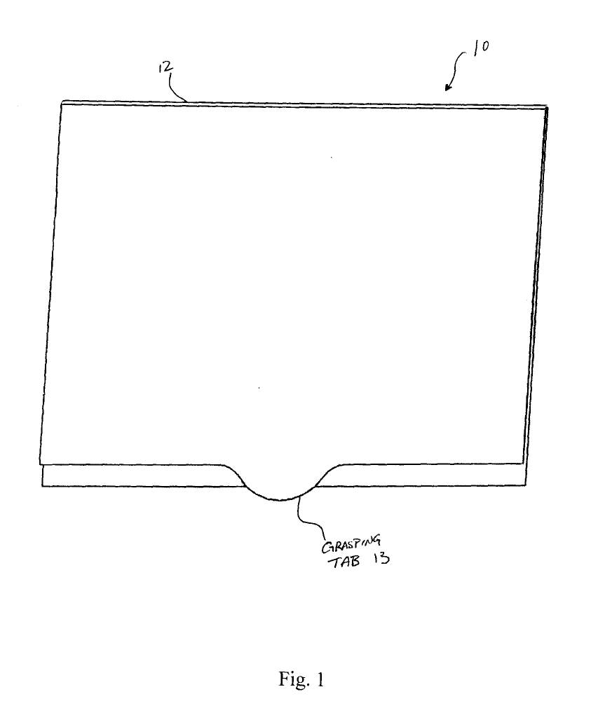

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the processes, methods, and systems for the delivery of fragrance or other applications of the present invention, as represented in FIGS. 1 through 7, is not intended to limit the scope of the invention, as claimed, but is merely representative of presently preferred embodiments of the invention.

Referring to FIG. 1, a greeting card 10 is shown with a single fold 12. In one embodiment, a grasping tab 13 facilitates opening the card 10. The present invention may be configured to work with cards or other products with opposing sides. One of skill in the art will appreciate that multiple fold cards can be configured to provide opposing sides. Furthermore, the present invention may work with a single face and detachable cover to the face such that a fold is not necessary.

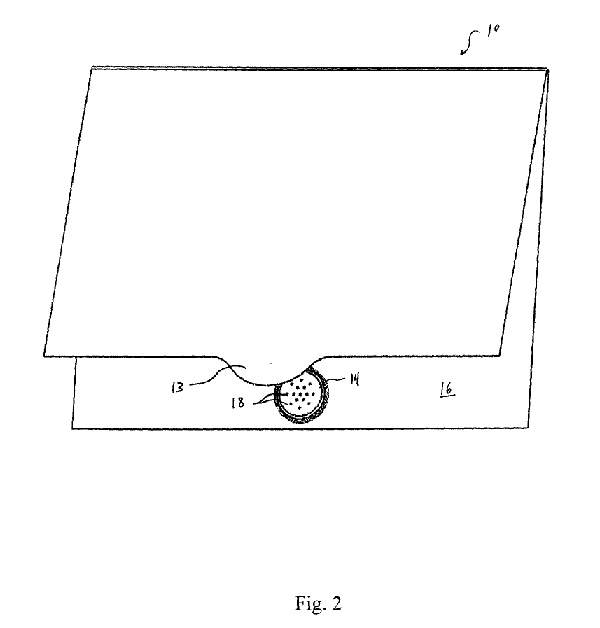

Referring to FIG. 2, the card 10 is shown partially open to reveal a canister 14 attached to a first face 16 of the card 10. The canister 14 is configured to receive a fragrance or fragrance-emitting material. The canister 14 may be thin metal discs crimped together and may contain one or more orifices 18 to allow fragrance to escape from the canister 14. The orifices 18 may be randomly or strategically spaced and sized according to a desired rate of escape of the fragrance from the canister 14. The fragrance-emitting material and/or emanator may be in the form of fluid, vapor, powder or other solid, a gel, particles suspending in any of the forgoing, a solution, and the like. As used throughout this specification, “canister” may be any item capable of containing, supporting, or attaching to, a fragrance-emitting material. For example, the canister may be a simple platform to which the fragrance-emitting material is bonded or connected. Further, “cover” may be any item which inhibits fragrance from emanating from the canister. For example the cover 20 may be dome-shaped to cover a flat surface canister 14, or it may be a plug or other arrangement to inhibit fragrance from moving away from the canister 14. Additionally, the term “fragrance” includes anything detectable by the sense of smell and need not necessarily only refer to pleasant smelling material. For example, in one embodiment, a humorous or “gag gift” card may contain fragrance-emitting material that is not pleasant smelling. Indeed, “fragrance-emitting material” includes all odor-emitting material, and “fragrance” includes all odor as used herein throughout.

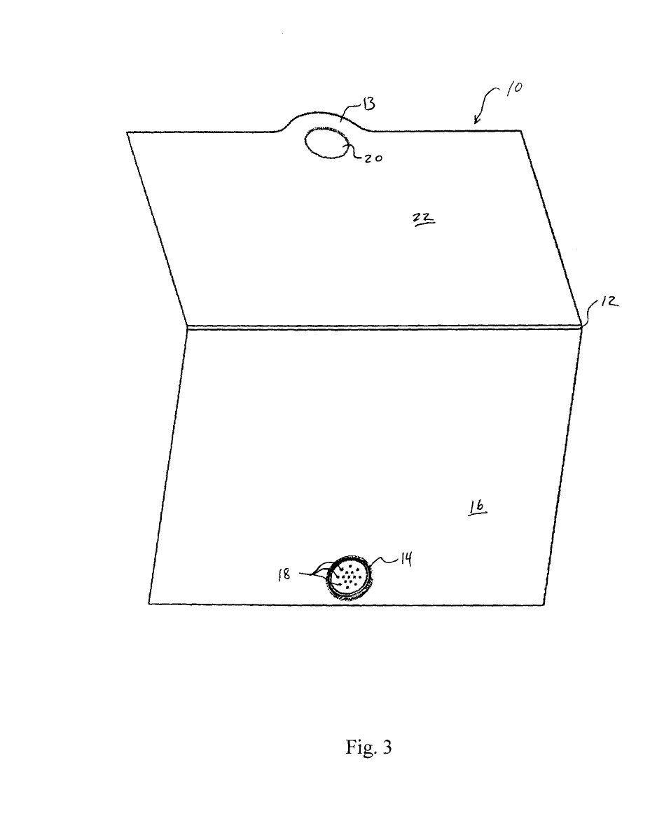

Referring to FIG. 3, the card 10 is more fully opened to reveal a cover 20 positioned or attached on or within a second face 22 of the card 10. In one embodiment, the cover 20 is attached to or positioned within the second face 22 such that when the card 10 is closed relative to the fold 12, the cover 20 covers one or more of the orifices 18 of the canister 14 attached to or positioned within the first face 16. The canister 14 and cover 20 may be positioned such that when the cover 20 is placed over canister 14 it substantially prevents or partially inhibits the fragrance-emitting material from escaping the canister 14. It will be appreciated by those of skill in the art that the canister 14 and cover 20 may be made from a variety of materials including plastics, rubbers, synthetic materials, metals alloys, combinations thereof, and the like. In one embodiment, the material of the canister 14 and cover 20 is inert to the fragrance-emitting material such that the material does not react with the canister 14 and/or cover 20. The cover and canister may also have a variety of configurations. The cover 20 may be several coverings corresponding to each hole of opening in the canister 20. For sealing purposes, the cover 20 and canister 14 may each have a substantially flat service aligned with each other. The openings of the canister 14 may be disposed within the flat surface allowing the cover 20 to be adjacent to the surface in which the openings are configured. The surface of the canister 14 may take other shapes and the cover 20 can be configured to match the shape of the canister to provide a good seal. It will be appreciated by those of skill in the art that an orifice includes any opening or passageway though which odor may pass.

In one embodiment, the cover 20 and the canister 14 are made of material such that when the cover 20 is in close proximity to the canister 14, a magnetic force attracts the cover 20 onto the canister 14. In one embodiment, the canister 14 and/or cover 20 may be a magnet or include a magnetic component. In one embodiment, the magnetic component is the material of the cover 20 and/or canister 14 that has been magnetized. One of skill in the art will appreciate that the size of the magnetic force between the cover 20 and canister can be predetermined to allow closure of the card 10 without requiring undue force by a user to open the card 10. In one embodiment, the cover 20 is a flexible magnet and the canister 14 is made of nickel plated steel. In another embodiment, the canister 14 is a flexible magnet and the cover 20 is made of nickel plated steel. It will be appreciated by those of skill in the art that the cover 20 and canister 14 can be configured in a variety of ways, with a variety of materials, to cause the magnetic attraction and provide for the covering of the canister 14.

It will be appreciated by those of skill in the art that the cover 20 may cover the orifices 18 of the canister 14 in a number of ways, including without limitation, by press fit, snap fit, latches, hook and loop arrangements such as Velcro, and other mechanical ways known in the art to secure a cover to a canister. Chemical or other means may also be utilized such as adhesives and the like. The cover 20 may attach to the canister 14 directly or indirectly. For example, areas of the first and second faces 16 and 22 of the card 10 may attach to each other in a way that positions the cover 20 over the orifices 18 of the canister 14, as will be discussed in greater detail below. In other embodiments, the surfaces surrounding the cover 20 and canister 14 attach to each other such that the cover is aligned with the canister. For example, the greeting card may have a Velcro arrangement such that opposing faces of the card attach to each other in a hook and loop arrangement, thus positioning the cover 20 over the canister 14. In another embodiment, magnets positioned adjacent the cover and/or canister cause the faces of a card to attach to one another such that the cover is positioned adjacent to the canister to inhibit or substantially prevent fragrance from escaping the canister.

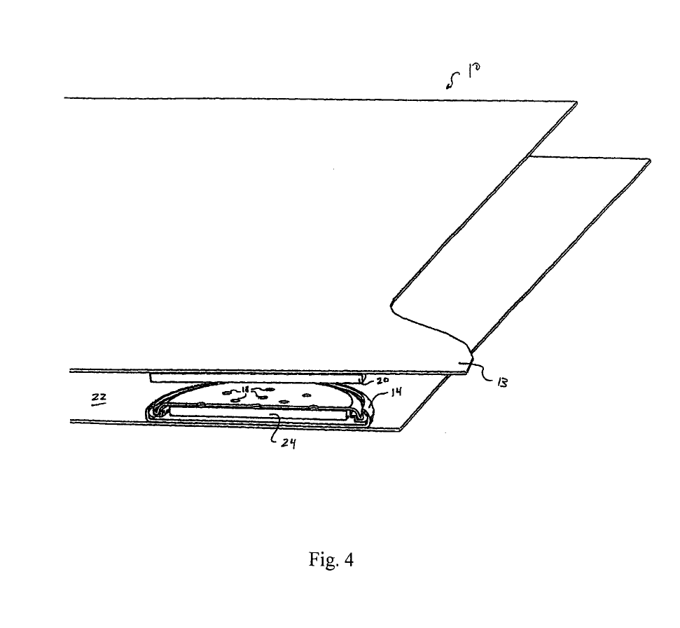

Referring now to FIG. 4, a cutaway view of the card 10, canister 14, and cover 20 is shown, revealing an emanator 24 within the canister 14. The emanator 24 may contain the fragrance-emitting material. In one embodiment, the fragrance-emitting material is a liquid. The emanator 24 may be an absorbent material such as paper, gelatinous substances, plastic, cellulous material and the like. In one embodiment, the emanator 24 is a porous material configured such that the surface tension and/or wicking action within the emanator 24 is equal to or greater than the force of gravity, which allows the emanator 24 to keep the fluid from dripping or seeping out. The emanator 24 in one embodiment may be the fragrance-emitting material. In other words, the structure which could house or support fragrance-emitting material may itself be fragrance emitting.

The choice and quantity of the fragrance-emitting material together with the configuration of the canister is such to allow fragrance to be released from the canister 14 during repeated removals of the cover 20 from the canister 14. It is anticipated that the canister 14 and emanator 24 can be configured to release fragrance after 500 openings or more. The canister 14 with the proper choice of fragrant material, could still be fragrant after one year. It will be appreciated by those of skill in the art that the combination of the cover 20 in operable communication with the canister orifices 18 may be such to prevent any fragrance-emitting material within the canister 14 from oxidizing over an extended period of time, even with frequent covering and uncovering of the canister orifices 18.

In one embodiment, the canister 14 contains a gas generating substance. The gas generating substance may be on or within the emanator 24. The gas generating substance may be an effervescent substance such that upon opening the canister 14 the material reacts with the moisture in the air to create a gas that acts as a carrier for the fragrance to escape the canister 14. In one embodiment the effervescent substance is dry citric acid mixed with a dry carbonate, including without limitation, calcium carbonate, sodium carbonate, and the like. Upon exposure to air the effervescent material will create carbon dioxide to help carry the scent within the canister outside. In another embodiment, the gas generated by the gas generating chemical in the canister 14 is hydrogen. In one embodiment, the substance may be a dry mixture with fragrance added to it or in close proximity to it so that the effervesced gas carries the fragrance with it.

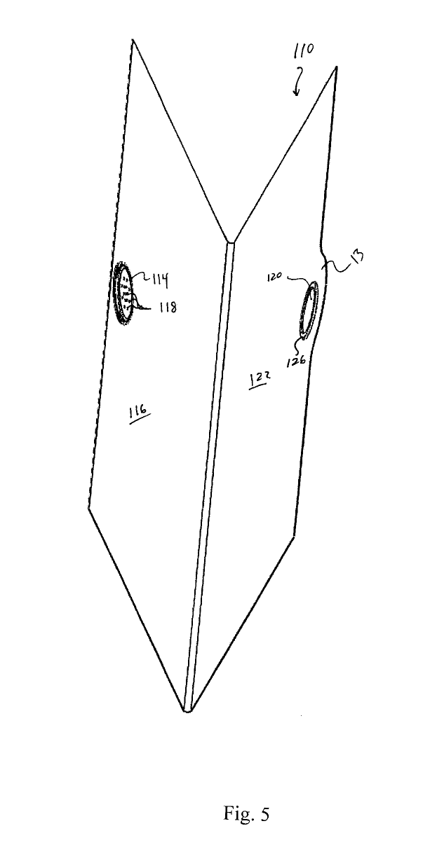

Referring now to FIG. 5, another embodiment of the present invention is shown. A first face 116 of a card 110 contains a canister 114 with orifices 118. A second face 122 of the card 110 contains a cover 120. An attachment member 126 is positioned adjacent the cover 120 to facilitate attachment of the first face 116 to the second face 122 and the positioning of the cover 120 over one or more orifices 118. It will be appreciated by those of skill in the art that the cover and/or canister may be attached to a respective face of a card in any number of ways including bonding, or other chemical means, Velcro, or other mechanical means, or the by the configuration of the card itself. In operation, of this and other embodiments, the opening of the card 110 removes the cover 120 from the canister allowing the fragrance to escape, or more fully escape the canister 114. This provides an olfactory experience to the user.

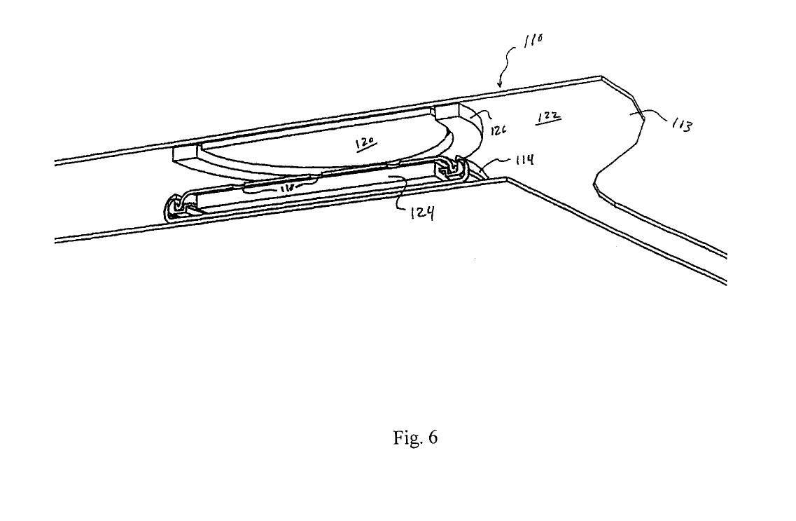

Referring now to FIG. 6, a cutaway view of the embodiment of FIG. 5 is shown. In this embodiment, the attachment member 126 is an annular magnet that attaches to the canister 114 when the card 110 is closed. It will be appreciated by those of skill in the art that the attachment member 126 may be in a variety of configurations utilizing a variety of attachment mechanisms as discussed above, including without limitation, a Velcro ring etc. In one embodiment, the cover 120 may be a compliant material sealed to face 122 at its edges such that upon closure of the card, the material confirms to the surface of the canister 114 containing the orifices 118 in order to substantially prevent fragrance from escaping the canister 114.

In the embodiment of FIG. 6, the cover 120 may itself be an emanator. When the card 110 is closed, the attachment member 126 magnetically connects to the canister 114 forming a chamber that substantially retains any fragrance there within. A liner (not shown) may be positioned between the emanator cover 120 such that fragrance-emitting material, vapor, or fragrance does not seep into the second face 122 of the card 110. The liner may be of any type of material known in the art that serves as a barrier. As the card 110 is opened, the emanator cover 120 moves through the air away from the canister 114 effectively mixing fragrance with the air and creating a small air current which may also help draw fragrance out of the canister 114. When closed, the opposing emanator pad cover 120 would be in close proximity to the canister 114 to absorb any vapor and carry it through the air upon the opening of the card 110. The opposing emanator cover 120 pad may be made of the same material as the canister emanator pad 124.

In another embodiment, there may be one or more large orifices 118 into which portions or all of the opposing emanator pad cover 120 may fit. Similarly, the opposing emanator pad cover 120 would be in close proximity to the emanator 124 in canister 114 to absorb any vapor and carry it through the air upon the opening of the card 110. In yet another embodiment, the canister 114 may contain a screen layer or mesh or porous material such as those discussed above, which would also allow fragrance to escape from the canister. The canister 114 may be any container suitable for retaining fragrance emitting material for at least a period of time.

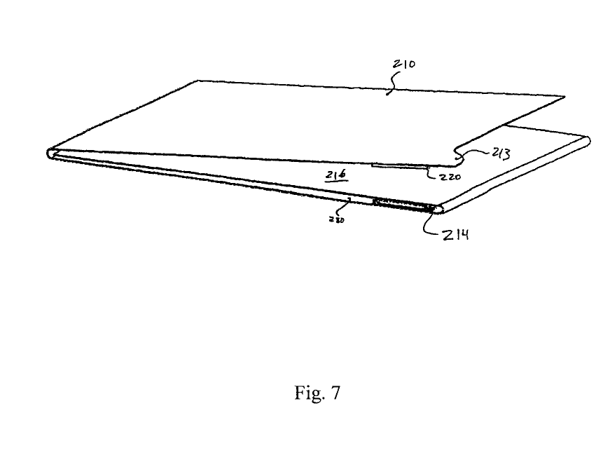

Referring now to FIG. 7, the cross section of another embodiment of the invention is shown. In this embodiment, a card 210 has multiple folds which allow the canister 214 and/or cover 220 to be hidden. In this embodiment, the first face 216 may have a portion adjacent the canister 214 with allows fragrance to pass there through. In one embodiment, the fragrance passes through the paper. In another embodiment, a material (not shown) may be loosely placed within an area 230 such that upon closure of the card 210, the material may press upon the orifices of the canister 214 within the area, substantially preventing the escape of fragrance from the canister 214. Upon the opening of the card 210, the material may be allowed to move and thereby allow fragrance to escape the canister 214.

Other members may be included in the canisters 14, 114, and 214 and or covers 20, 120, and 220 discussed herein to facilitate the escape of fragrance from the canisters 14, 114, 214. These members may include a gas generating cell to pump the fragrance out, a heater, a fan, and the like. These members may also include substances in the canister to stir or agitate the fragrance-emitting material to encourage the release of the fragrance from the canister 14, 114, and 214. One such agitation member may be a magnetic powder such as Fe₃O₄ or other magnetic powders mixed into a fragrance powder such that the presence of a magnetic cover 20, 120, or 220 may attract the powder thereby stiffing the fragrance-emitting material.

One of skill in the art will appreciate the canister 14, 114, and 214, and cover 20, 120, and 220, may be configured in a variety of ways to prevent the escape of fragrance from the canister 14, 114, and 214 when the card is in a closed position and allow the escape of fragrance from the canister 14, 114, and 214 when the card is in an open position.

While specific embodiments of the present invention have been illustrated and described, numerous modifications come to mind without significantly departing from the spirit of the invention, and are included within its scope. The scope of the invention is defined by the following claims.

1. A fragrance delivery system, comprising:
a canister attached to a first surface;
an emanator disposed within the canister, the emanator comprising a fragrance-emitting material;
a removable cover attached to a second surface such that the when the first and second surface are brought together, the cover inhibits or prevents fragrance from escaping the canister.

2. The system of claim 1, wherein the canister comprises at least one orifice disposed within the canister.

3. The system of claim 2, wherein the cover covers the at least one orifice disposed within the canister.

4. The system of claim 1, wherein the first and second surfaces comprise opposing faces of a card.

5. The system of claim 4, wherein the first or second surfaces comprise a grasping tab.

6. The system of claim 1, wherein the canister is substantially flat.

7. The system of claim 1, wherein the cover comprises a magnetic component.

8. The system of claim 7, wherein the cover comprises nickel plated steel.

9. The system of claim 1, wherein the canister comprises a magnetic component.

10. The system of claim 9, wherein the canister comprises nickel plated steel.

11. The system of claim 1, wherein the material of the canister and cover are inert to the fragrance-emitting material such that the canister and cover do not react with the fragrance-emitting material.

12. The system of claim 1, wherein the cover comprises material chosen from plastic, rubber, synthetic material, metals alloys, and combinations thereof.

13. The system of claim 1, wherein the emanator comprises a fluid.

14. The system of claim 1, wherein the emanator comprises a porous solid.

15. The system of claim 14, wherein the emanator comprises paper.

16. The system of claim 1, wherein the emanator comprises a gelatinous substance.

17. The system of claim 1, wherein the canister contains a gas-generating substance.

18. The system of claim 17, wherein the gas-generating substance comprises a carbonate.

19. The system of claim 17, wherein the gas-generating substance comprises citric acid.

20. The system of claim 1, wherein the canister comprises an agitation member.

21. The system of claim 20, wherein the agitation member comprises Fe₃O₄.

22. The system of claim 1, further comprising a gas generating cell.

23. A fragrance delivery system, comprising:
a canister attached to a first surface of a card, the canister defining orifices;
a gas generating substance disposed within the canister, such that when the gas-generating substance is exposed to air outside the canister, the gas-generating substance emits a fragrance;
a cover attached to a second surface of a card such that the when the first and second surfaces are brought together, the cover inhibits or substantially prevents the fragrance from escaping the canister.

24. The system of claim 23, wherein the canister comprises an agitation member.

25. The system of claim 24, wherein the agitation member comprises Fe₃O₄.

26. A fragrance delivery system, comprising:
a canister comprising nick plated steel attached to a first surface of a card, the canister defining orifices;
an emanator disposed within the canister, the emanator comprising a fragrance-emitting material;
a cover comprises a magnetic component attached to a second surface of a card such that when the first and second surface are brought together, the cover substantially prevents a fragrance from escaping the canister;
a cover attached to a second surface of a card such that the when the first and second surface are brought together, the cover magnetically connects to the canister and inhibits the fragrance from escaping the canister; and
an agitation member comprising Fe₃O₄.
